(19) United States
(12) Patent Application Publication
(10) Pub. No.: US 2005/0261780 A1
(43) Pub. Date: Nov. 24, 2005

(54) Form-fitting bioabsorbable mesh implant

(76) Inventors: Harri Heino, Tampere (FI); Kari Laakso, Tampere (FI); Tero Valimaa, Tampere (FI); Andreas Jaschek, Jalisco (MX); Pertti Tormala, Tampere (FI)

Correspondence Address: KENYON & KENYON, 1500 K STREET NW, SUITE 700, WASHINGTON, DC, 20005, US

(21) Appl. No.: 09/876,065

(22) Filed: Jun. 8, 2001

(51) Int. Cl. A61F 2/28

(52) U.S. Cl. 623/23.51; 623/23.58; 623/908

(57) ABSTRACT

The present invention provides a bioabsorbable, polymeric mesh implant for the fixation of bone fragments and bridging of bone defects or gaps. The bioabsorbable, polymeric mesh includes a plurality of openings and connectors, where each opening is connected to another opening by a connector. In addition, the bioabsorbable polymeric mesh is deformable at room temperature without breaking. The present invention also include embodiments drawn to methods of using the bioabsorbable, polymeric mesh implant. In an embodiment of the present invention, the method includes applying the bioabsorbable, polymeric mesh implant to a damaged bone area, the damaged bone area, which is curved, concave, convex, angular, spherical, or any combination thereof. Another embodiment of the present invention, substitutes the bioabsorbable, polymeric mesh implant including a film, for the mesh.

### FIELD OF THE INVENTION

The present invention relates to body tissue fixation systems, including body tissue fixation hardware comprising biocompatible, bioabsorbable (resorbable) polymeric meshes, and methods of using these systems and hardware.

### BACKGROUND OF THE INVENTION

Traditional orthopedic and traumatological fixation systems to facilitate bone fracture healing (osteosynthesis) typically employ metallic hardware, (e.g. plates, screws rods and the like), formed of biocompatible, corrosion resistant metals such as titanium, and stainless steel. Examples of typical metallic plates are described in the book, F. Sequin and R. Texhammnar, AO/ASIF Instrumentation Springer-Verlag, Berlin. Heidelberg. 1981, at p. 21-22, 55-79, 107-108. 117-122, the disclosure of which, is incorporated herein by reference in its entirety.

In maxillofacial and in cranial surgery, metallic mini plates are popular for use. See e.g., W. Muhlbauer et al., Clin. Plast. Surg. 14 (1987) 101-111; A. Sadove and B. Eppleg. Ann. Plast. Surg. 27 (1991) 36-43; and R. Suuronen, Biodegradable Self-reinforced Polylactide Plates and Screws in the Fixation of Osteotomies in the Mandible, Doctoral Thesis, Helsinki University, Helsinki, 1992, p. 16, the disclosure of which is incorporated herein by reference, in its entirety. Mini plates are small, thin, narrow plates, which have holes for screw fixation. The mini plates are typically located on bone, perpendicular to the fracture, to secure the bone mass on both sides of the fracture to each other. Typical geometry's of mini plates are described in U.S. Pat. No. 5,290,281, the entire disclosure of which is incorporated herein by reference in its entirety.

While such systems are generally effective for their intended purposes, they possess a number of inherent shortcomings. First, metal release into the surrounding tissues has been reported. See, e.g., L.-E. Moberg et al. Int. J. Oral. Maxillofac. Surg. 8 (1989) at p. 311-314 the disclosure of which is incorporated herein by reference in its entirety. Second, stress shielding as described by, P. Paavolainen et al. Clin Orthop. Rel. Res. 136 (1978) 287-293, the disclosure of which is incorporated herein by reference, in its entirety, has also been observed.

Finally, growth restriction in young individuals as detailed in K. Lin et al Plast. Reconstr. Surg. 87 (1991) 229-235, the entire disclosure of which is likewise incorporated herein by reference, in its entirety, has also been a problem. For example, as described in J. Fearon et al Plast. Reconstr. Surg. 4 (1995) 634-637, the disclosure of which is incorporated herein by reference, in its entirety, there is the risk that metallic plates and screws can sink into and below the cranial bone in infants and young children as a consequence of skull bone growth, thereby threatening the brain. Therefore, it is generally recommended that nonfunctional implants should be eventually removed, at least in growing individuals. See C. Lindqvist, Brit. J. Oral Maxillofac. Surg. 33 (1995) p. 69-70, the disclosure of which is incorporated herein by reference in its entirety.

Fixation plates have also been formed from bioabsorbable polymers. Even though these rigid plates can be deformed at room temperature, shaping these plates to fit a concave, convex, or spherical bone surfaces (e.g. cranium) bone surface is impossible without lessening the strength of the plate, (e.g. by cutting them into narrow sections or making radial cuts from the middle of the plate towards the edges of the plate). Narrow plate sections or radial cut plates do not support a plurality of bone fractures as well as one continuous implant. To achieve sufficient deformation behavior and still have enough rigidity and toughness to fix a plurality of bone fractures securely to their positions until the bone is healed, requires a special plate geometry.

One metallic device, which attempts to address the problem with solid plates is U.S. Pat. No. 5,468,242, the disclosure of which, is hereby incorporated by reference in its entirety. In U.S. Pat. No. 5,468,242, the metallic fixation device has increased three-dimensional deformability due to a special geometry. The metallic fixation device includes a plurality of fastening openings connected to each other by curved arms. However, this metallic device suffers from the same disadvantages of the previous metallic plates in that they would need to be removed after bone healing and would likely cause metal release to the surrounding tissues, stress shielding and growth restriction in young individuals.

A need therefore exists for bioabsorbable (bioresorbable or biodegradable) osteosynthesis fixation devices, which is strong, tough, does not produce a substantial inflammatory response, and which device can easily be deformed repeatedly in three dimensions. The devices must also be dimensionally stable in operating room conditions (e.g. in a first thermo-chemical state) to allow for fixation on large bone defects or a plurality of bone fragments on spherical surfaces like the cranium, without distortion of the configuration of the bone fragments to be fixed. The device must also be dimensionally stable in tissue conditions (e.g. at a second thermo-chemical state), when fixed on a bone surface to facilitate problem free bone fracture healing.

A need further exists for such bioabsorbable (bioresorbable or biodegradable) osteosynthesis devices, which is strong, tough, does not produce a substantial inflammatory response, and in which the openings used by fasteners maintain their original size and form during the deformation of the device in the first thermo-chemical state, to fit exactly on spherical, concave and convex bone surfaces.

A need also exists for such bioabsorbable (bioresorbable or biodegradable) osteosynthesis devices, which is strong, tough, does not produce a substantial inflammatory response, and whose deformation requires significantly less force, than the deformation of prior art bioabsorbable devices when fit to concave, convex and spherical bone surfaces.

Likewise, a need exists for such bioabsorbable (bioresorbable or biodegradable) osteosynthesis devices, which is strong, tough, does not produce a substantial inflammatory response, and which can easily be cut with e.g. normal scissors in the operating room under normal conditions when specific dimensions are needed for individual operations.

### SUMMARY OF THE INVENTION

The present invention provides a bioabsorbable, polymeric mesh implant for the fixation of bone fragments and bridging of bone defects or gaps. The bioabsorbable, polymeric mesh includes a plurality of openings and connectors, wherein each opening is connected to another opening by a connector. In addition, the bioabsorbable polymeric mesh is deformable at room temperature without breaking.

Another embodiment of the present invention also provides a bioabsorbable polymeric mesh implant for the fixation of bone fragments and bridging of bone defects or gaps. This embodiment of the present invention includes a bioabsorbable polymeric mesh comprising a plurality of openings and connectors, where each opening is connected to another opening by a connector and where the mesh has a first and second surface and a bioabsorbable film attached along either the first or second surface of the mesh. This embodiment of the present invention is also deformable at room temperature without breaking.

The present invention also includes embodiments drawn to methods of using the bioabsorbable, polymeric mesh implant. In an embodiment of the present invention, the method includes applying the bioabsorbable, polymeric mesh implant to a damaged bone area, the damaged bone area being curved, concave, convex, angular, spherical, or any combination thereof. Another embodiment of the present invention substitutes the bioabsorbable, polymeric mesh implant including a film, for the mesh.

Another embodiment of the present invention includes a method of using the bioabsorbable, polymeric mesh implant where the implant is deformed prior to applying it.

### BRIEF DESCRIPTIONS OF THE FIGURES

FIG. 1 shows a human skull with the bioabsorbable, polymeric mesh implant, according to the present invention fastened on the forehead.

FIG. 2 shows a human skull with the bioabsorbable, polymeric mesh implant, according to the present invention fastened on the forehead with bioabsorbable fasteners.

FIGS. 3A-3F show possible bioabsorbable polymeric mesh implant geometry's, according to the present invention.

FIG. 4 shows a bioabsorbable polymeric mesh implant of the present invention and a plate made from the same material, both deformed into a spherical form.

FIGS. 5A-5J shows examples of different connectors that can be used to connect the openings of the bioabsorbable polymeric mesh implant of the present invention.

FIG. 6A shows FEM modeling of a prior art design for a connector used in metal meshes showing the stress in the connector during deformation.

FIGS. 6B-6K show examples of FEM modeling of different types of connectors used in the bioabsorbable polymeric mesh implant of the present invention showing the stresses in the connectors during deformation.

FIG. 7A shows FEM modeling of a prior art plate.

FIG. 7B shows FEM modeling of a bioabsorbable polymeric mesh implant of the present invention.

### DETAILED DESCRIPTIONS OF THE FIGURES

The present invention provides a bioabsorbable, polymeric mesh implant, which can be easily deformed at room temperature. The bioabsorbable polymeric mesh implant includes a pattern of openings connected to each other by connectors, which can either be stretched or compressed during deformation, prior to implantation, without deforming the openings. The bioabsorbable polymeric mesh implant, provided by the present invention can be used on any area of a human skull to cover holes, to bridge two or more bone segments, to secure a plurality of bone fractions, to guide bone growth, or to heal any other type of bone injury. The bioabsorbable polymeric mesh can be attached to bone surfaces using bioabsorbable fasteners. And because cutting and deforming of the bioabsorbable polymeric mesh is easy to perform in operating room conditions, (e.g. there is no need for any heating equipment or special cutting devices), the total operating time is significantly reduced.

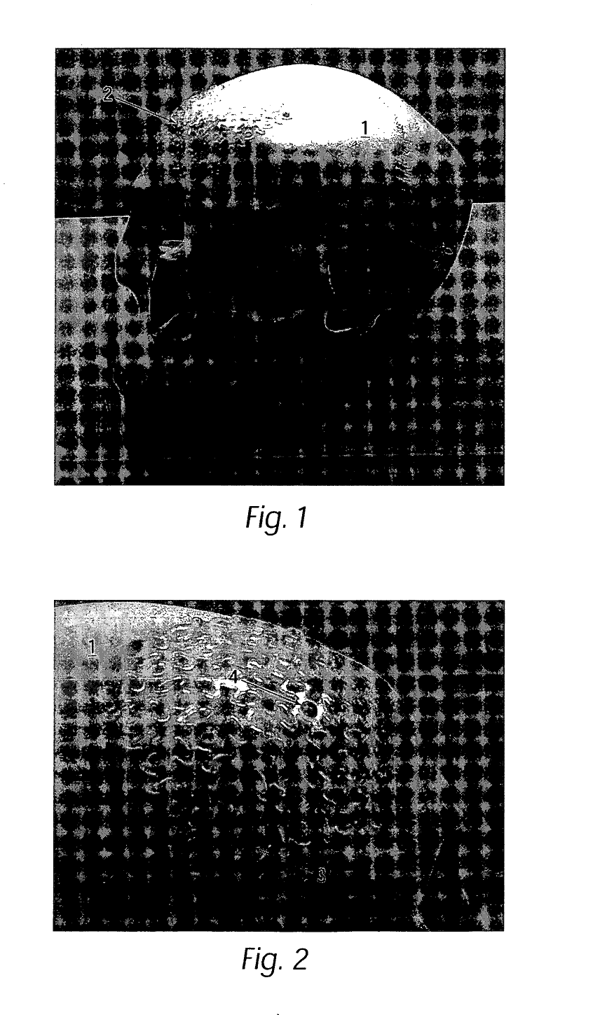

FIGS. 1 and 2 show the bioabsorbable polymeric mesh 2 of the present invention, fastened on the forehead of a human skull 1. In FIG. 2 the openings 3 of the bioabsorbable polymeric mesh implant are visible as are a few bioabsorbable fasteners 4 used to attach the bioabsorbable polymeric mesh 2 to the skull 1. The form-fitting, bioabsorbable implant, according to the present invention, can be used on the area of a human skull e.g. to cover holes, to bridge two or more bone segments, to secure plurality of bone fractions or to guide bone growth.

The osteosynthesis bioabsorbable, polymeric mesh of the present invention can be manufactured from malleable, biocompatible, bioabsorbable, strong and tough polymer materials, which can be unoriented, uni- or/and biaxially oriented. A non-exhaustive list of materials includes biocompatible, bioabsorbable, copolymers, polymer alloys, and composites. Examples of these types of biocompatible, bioabsorbable materials include, poly-α-hydroxy acids and other aliphatic bioabsorbable polyesters, polyanhydrides, polyorthoesters, polyorganophosphatzenes, tyrosine polycarbonates and other bioabsorbable polymers disclosed in numerous publications, see, e.g. S. Vainionp\ddot{aa} et. al., Prog. Polym. Sci., 14 (1989) 679-716, F1 Patent No. 952884, F1 Patent No. 955547 and WO-90104982, EP 0449867 B1, U.S. Pat. No. 5,569,250, S. I. Ertel et al., J. Biomed. Mater, Res., 29 (1995) 1337-1348, the disclosures of which is incorporated herein by reference, in its entirety.

The bioabsorbable, polymeric mesh can also be reinforced with reinforcing material such as fibres manufactured of a resorbable polymer or of a polymer alloy, or with biodegradable glass fibres, such as β-tricalsiumphosphate fibres, bio-glassfibres or CaM fibres. See for comparision, EP146398, the disclosure of which is incorporated herein by reference, in its entirety.

The open structure of the bioabsorbable, polymeric mesh enables selective transport of components, such as liquid and cells easily through the bioabsorbable, polymeric mesh. Bioactive reagents can be added to the bioabsorbable, polymeric mesh to help heal a given injury. In an embodiment of the present invention, different ceramic powders can be used as additives or fillers in bioabsorbable, polymeric mesh implants to promote new bone formation. Also drugs can be used as additives, such as antibiotics to suppress infections or anti-inflammatory agents to suppress inflammatory reactions caused by a trauma or by mesh absorption.

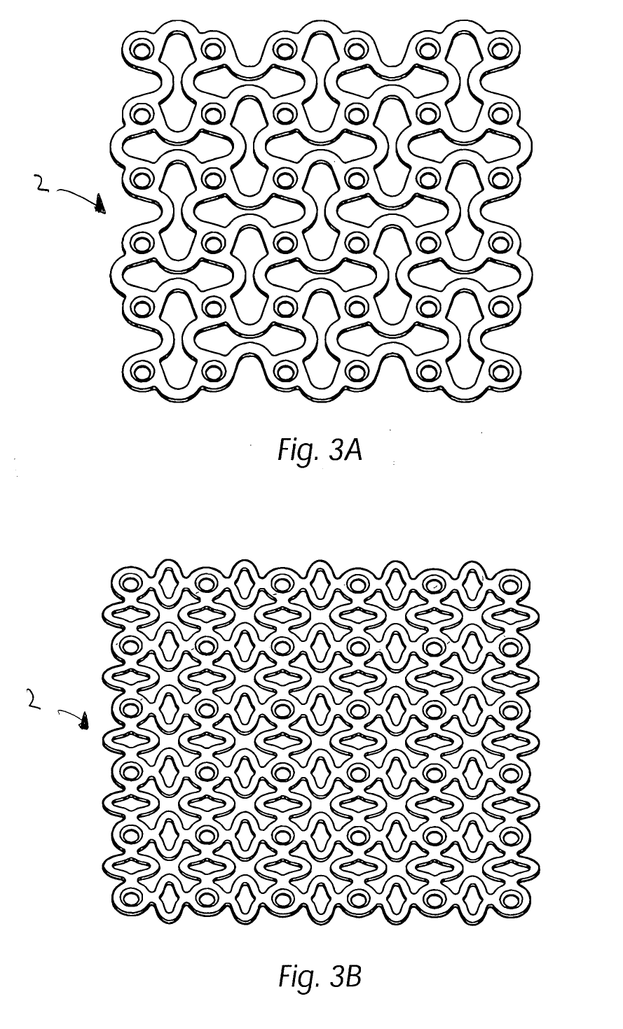

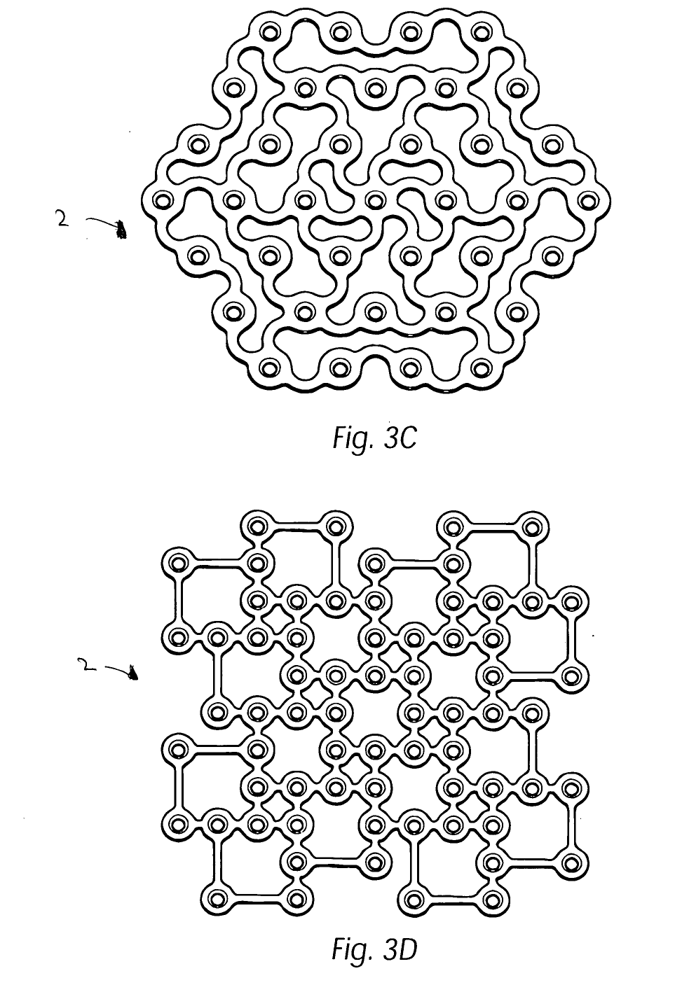

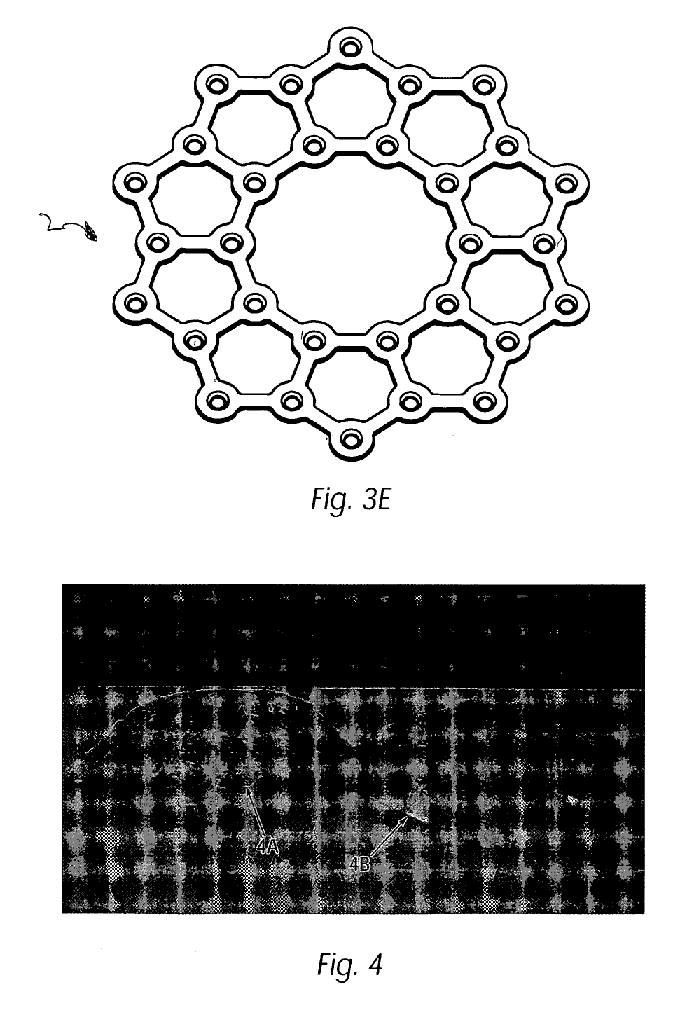

The structure of the bioabsorbable, polymeric mesh 2 shown in FIG. 2 includes an array of openings 3 connected to each other by connectors. The openings and connectors can form various mesh geometry's. FIGS. 3A-F show various embodiments of different bioabsorbable, polymeric mesh geometry's, which can be used to achieve sufficient deformation behavior. The embodiments shown in FIGS. 3A-E all have openings, which do not significantly deform during the deformation of the bioabsorbable, polymeric mesh implant. To attach the bioabsorbable, polymeric mesh of the present invention to bone surfaces, any type of bioabsorbable fastener can be used, including screws and tacks or the bioabsorbable, polymeric mesh can be sutured. When securing the bioabsorbable, polymeric mesh to bone with bioabsorbable fasteners all or only some of the openings need to be used.

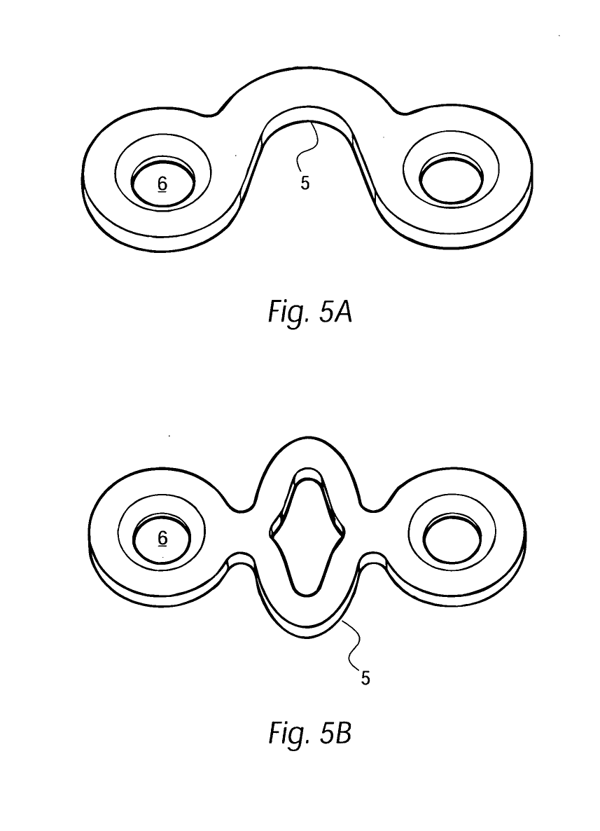

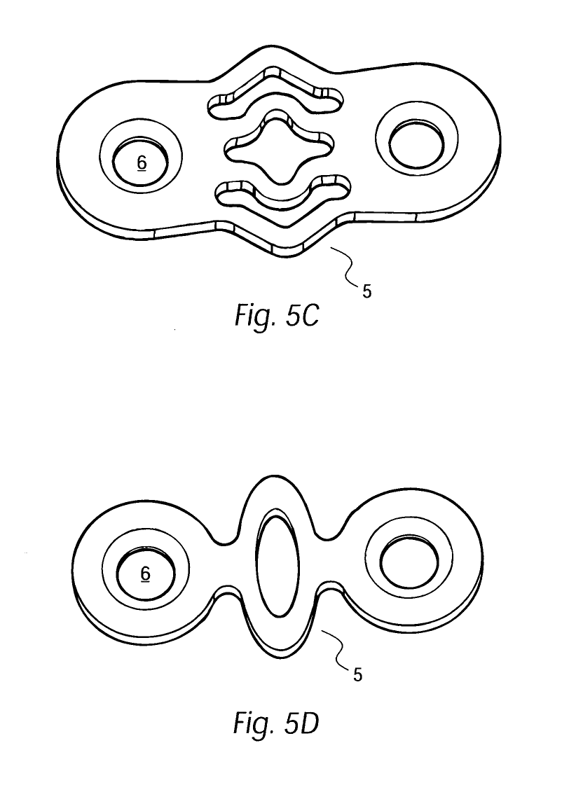

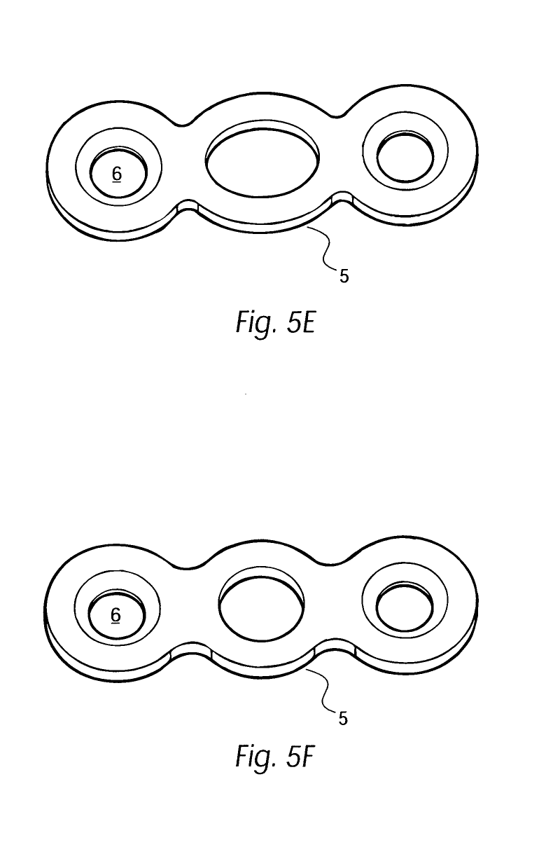

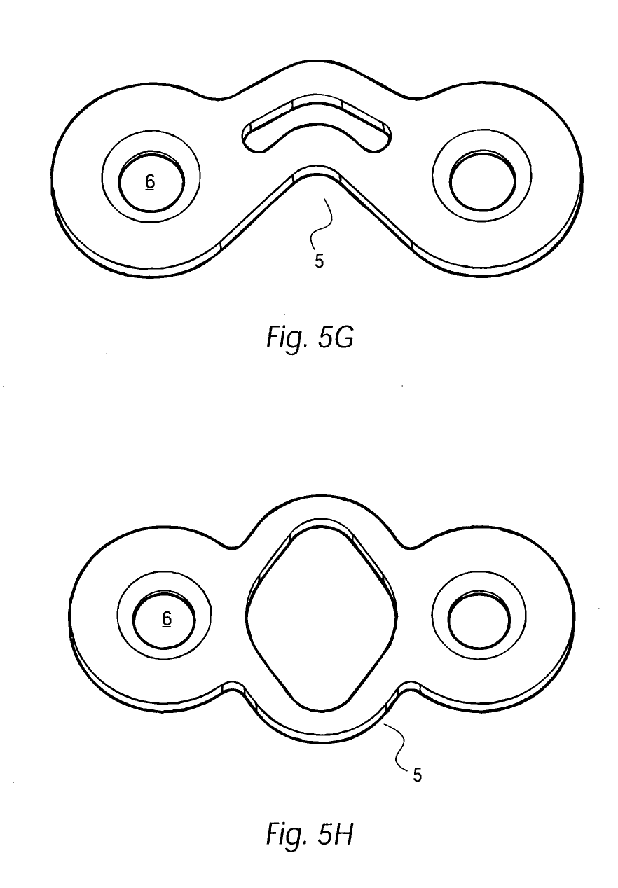

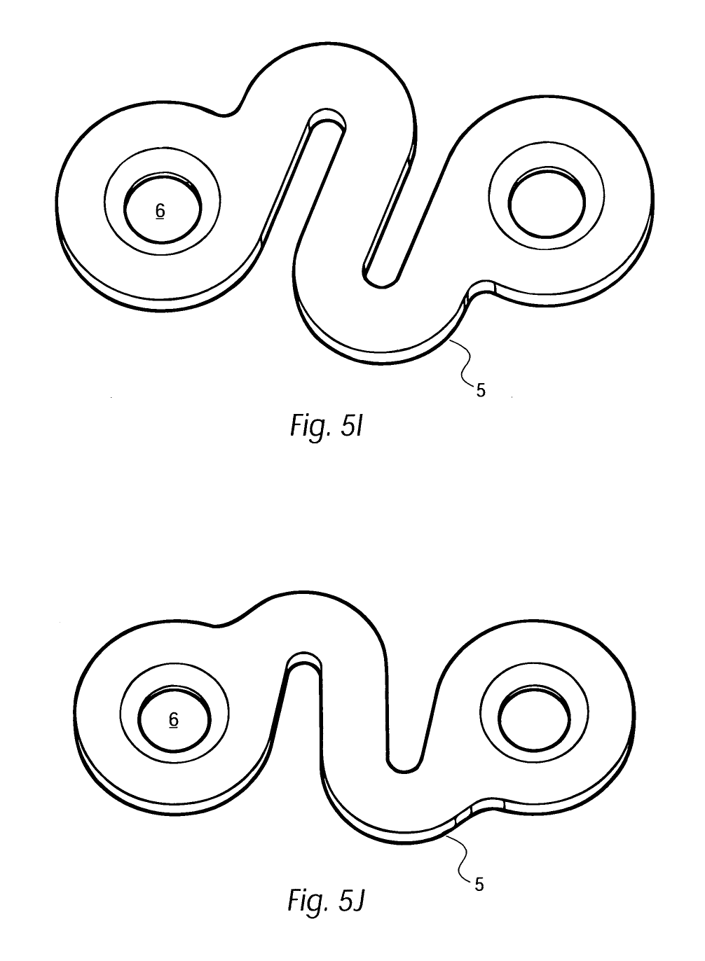

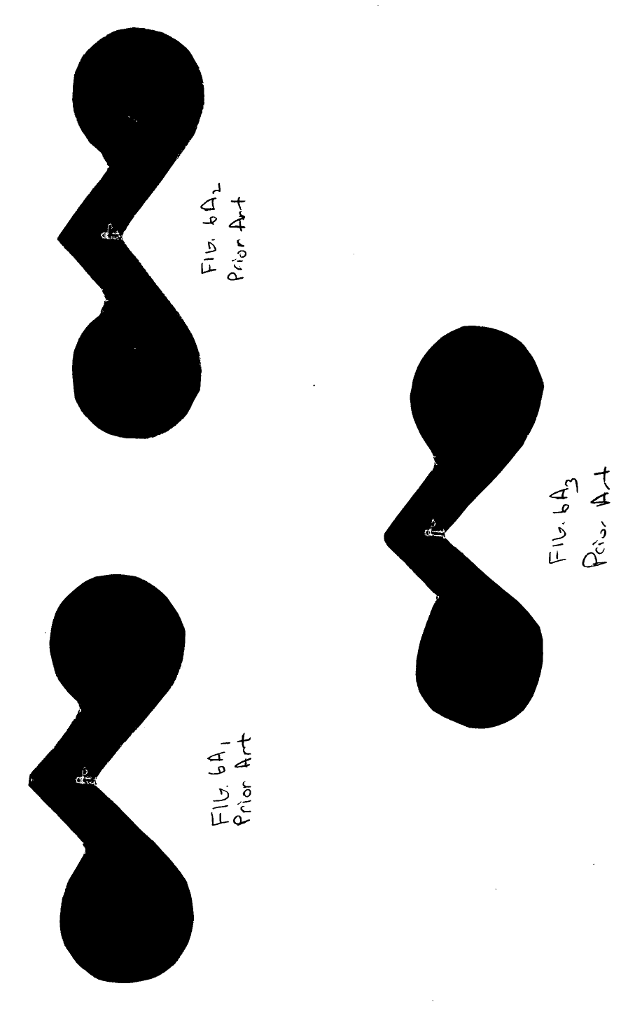

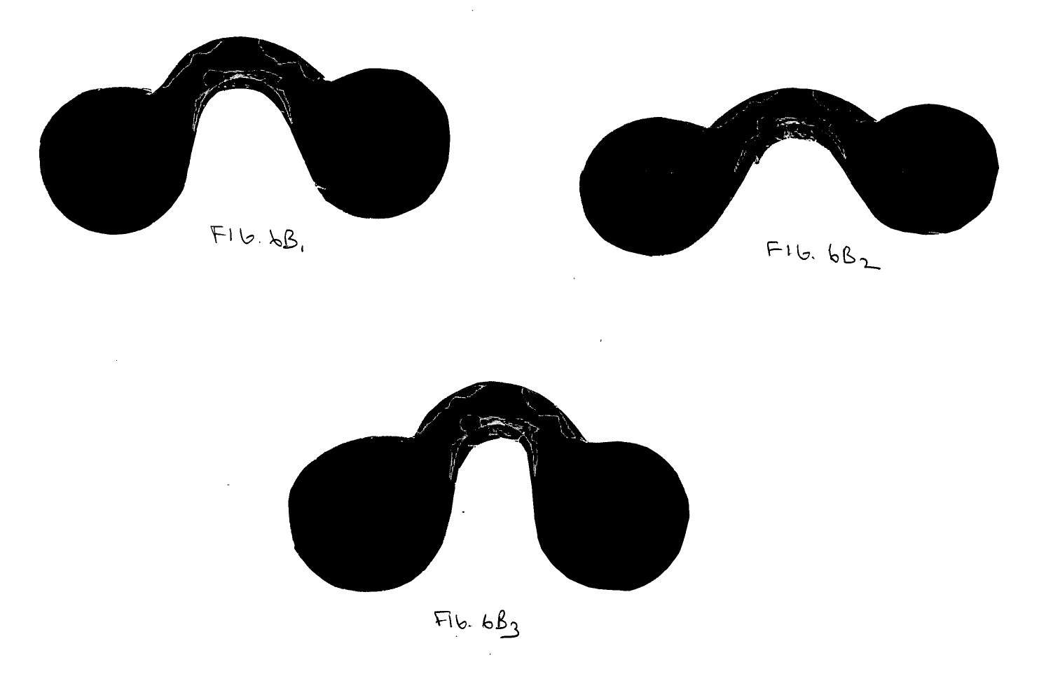

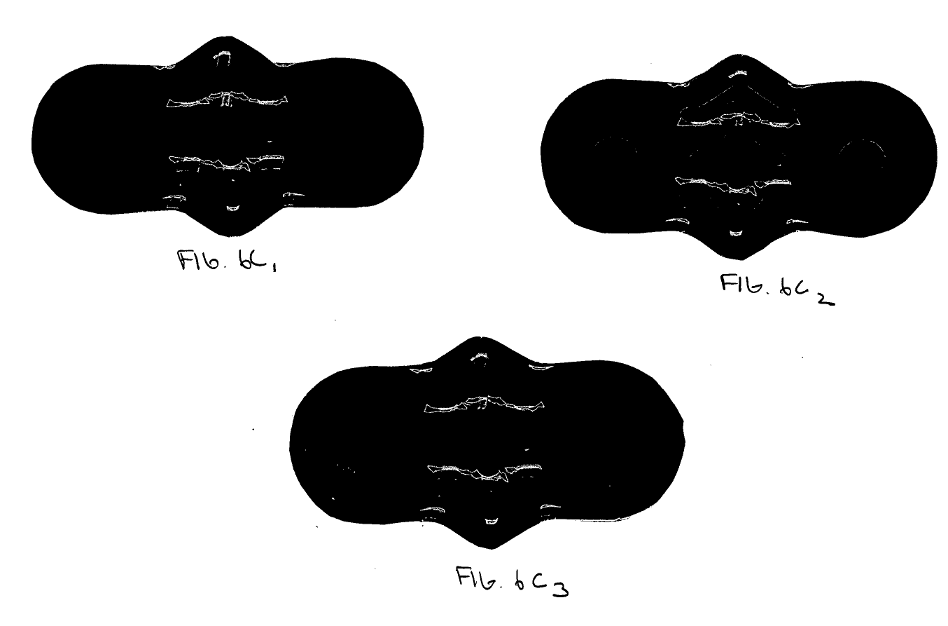

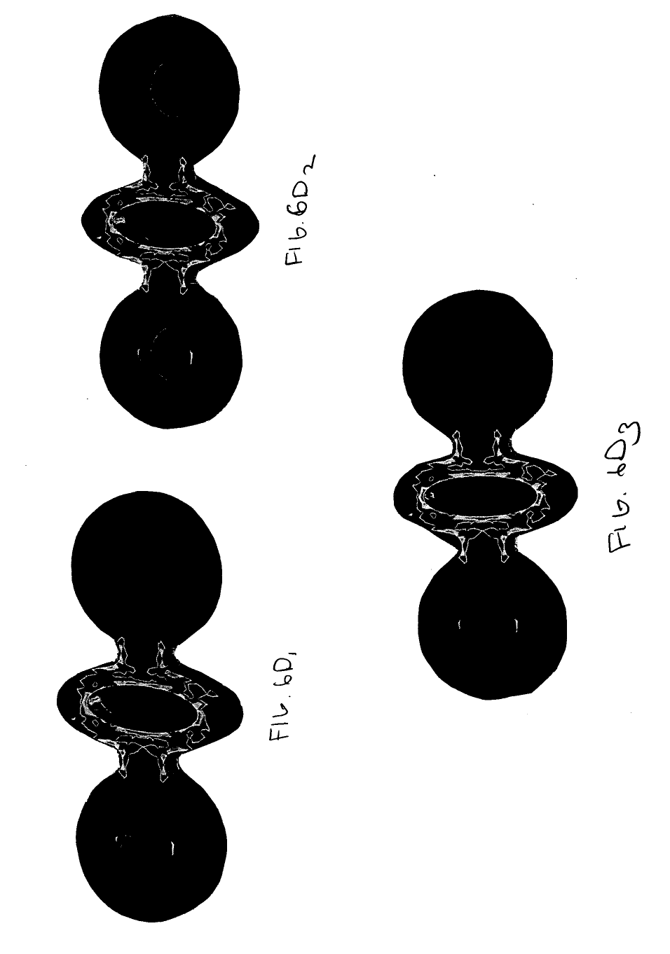

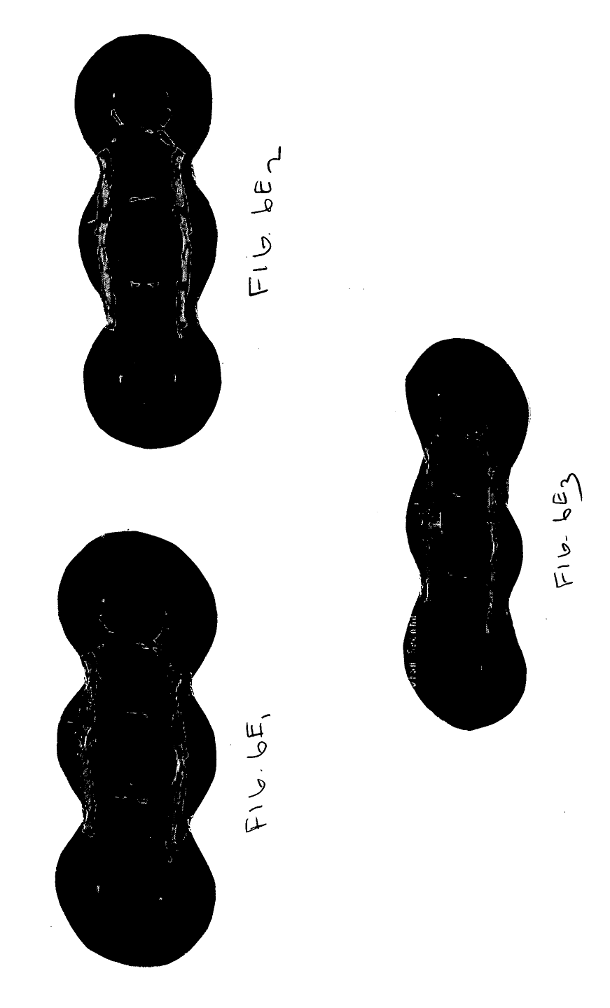

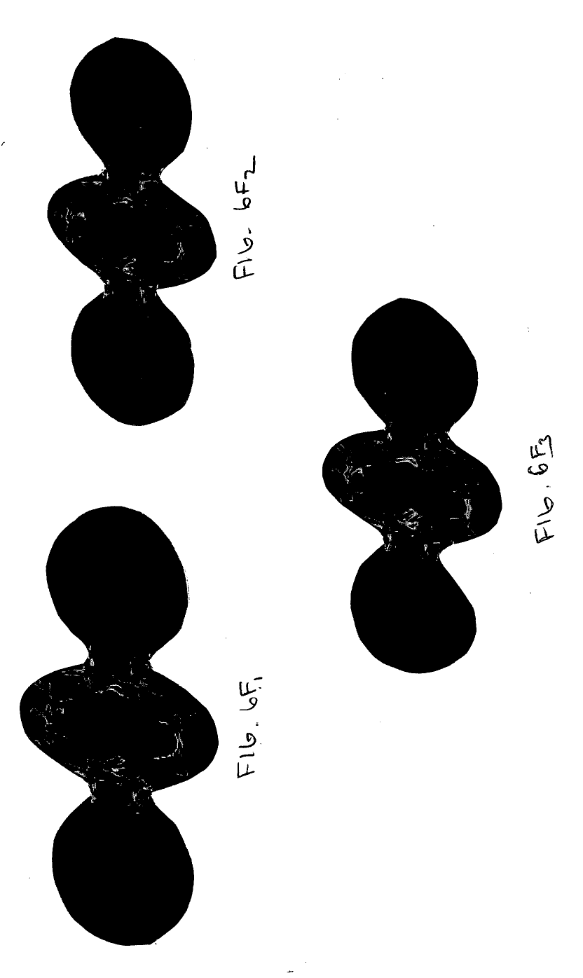

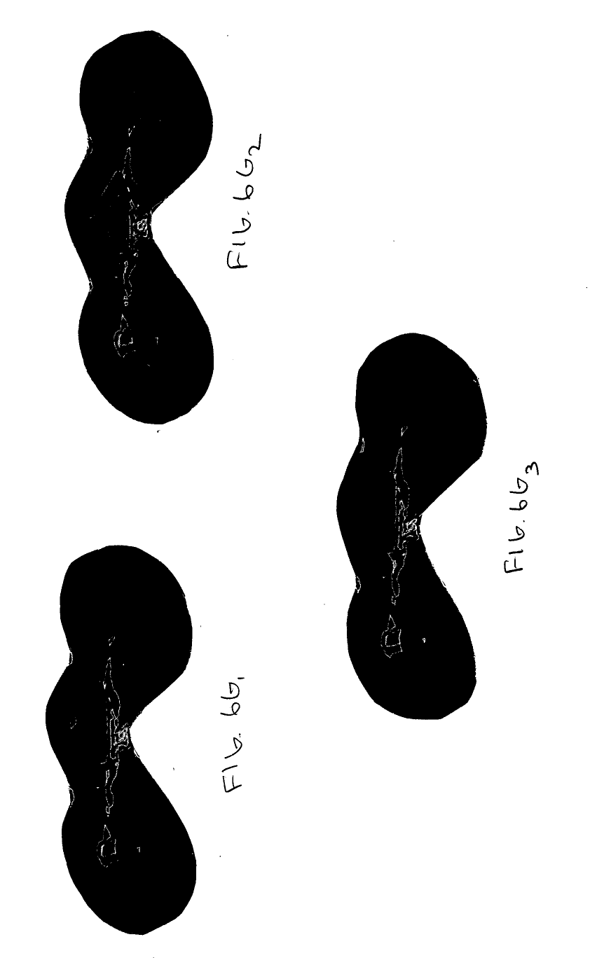

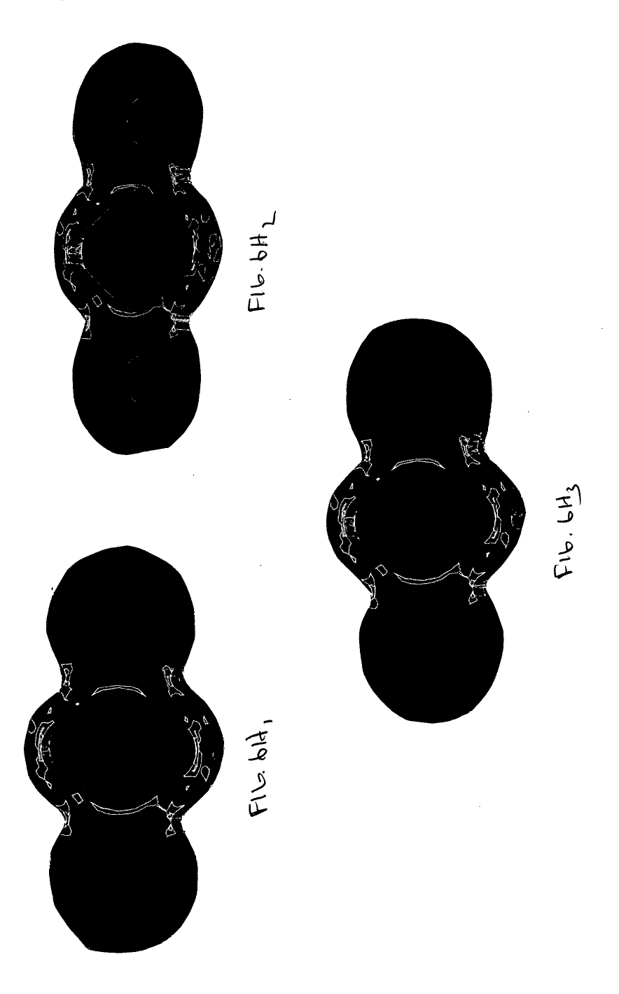

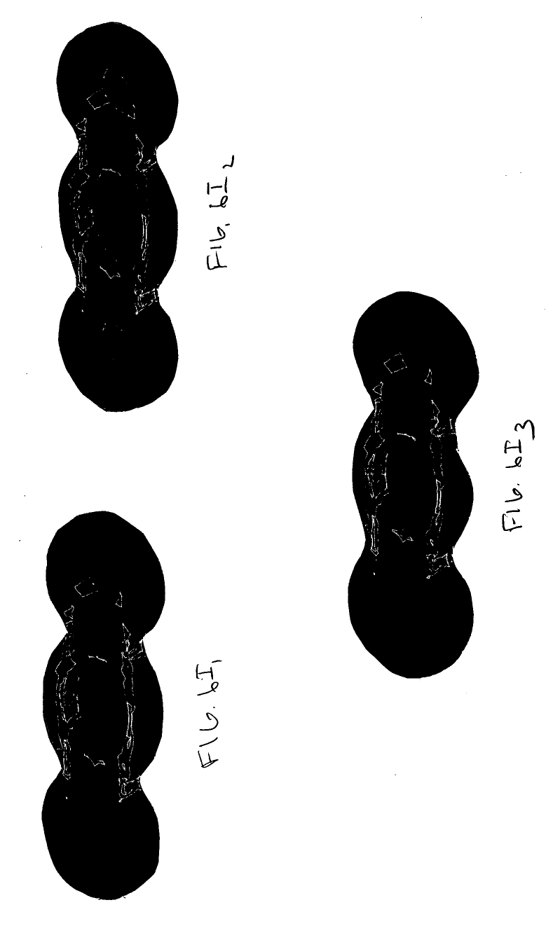

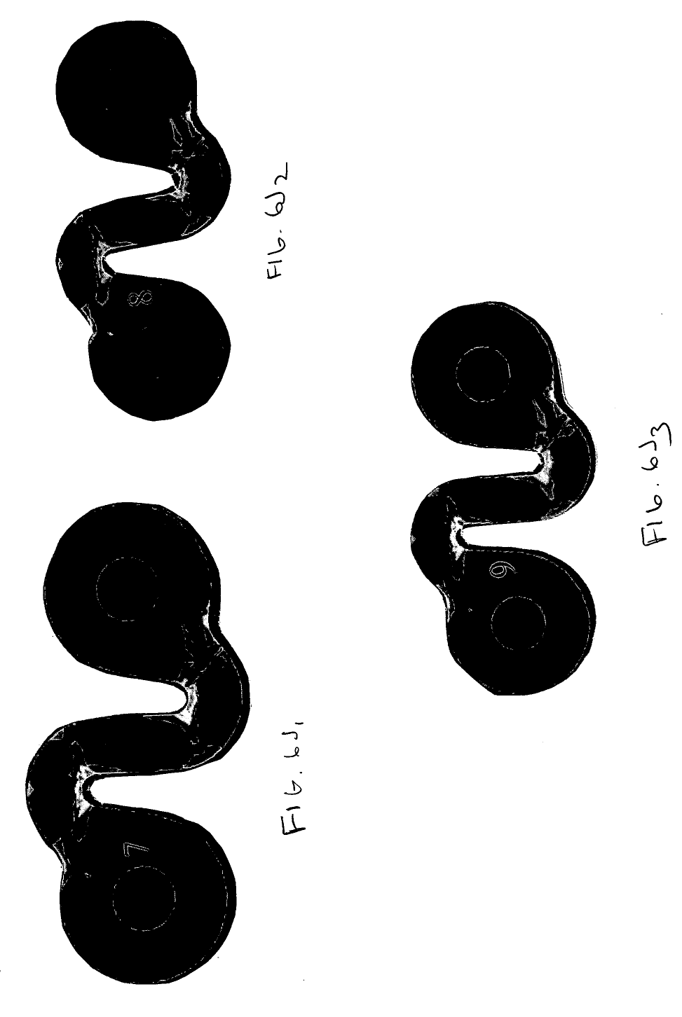

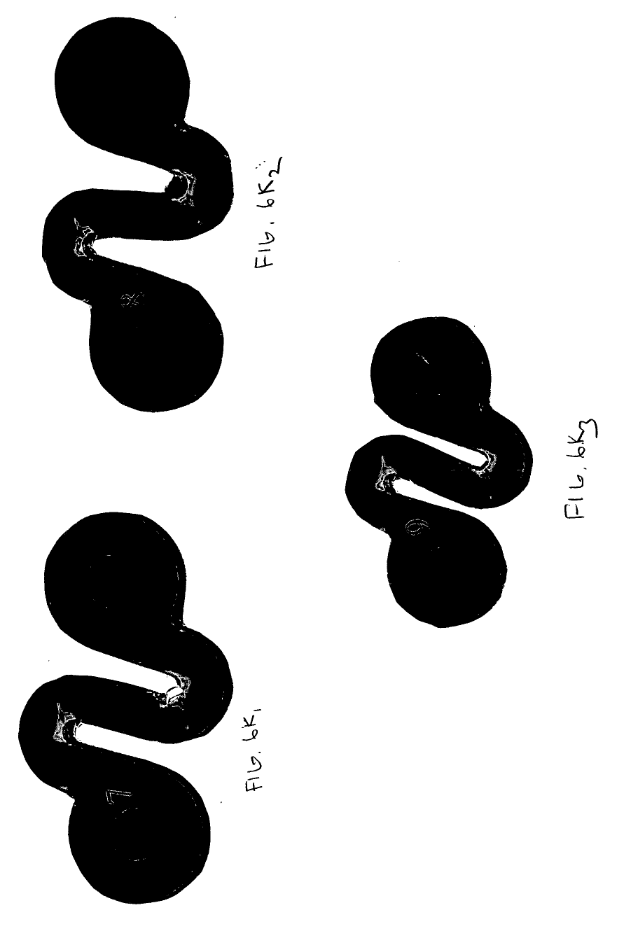

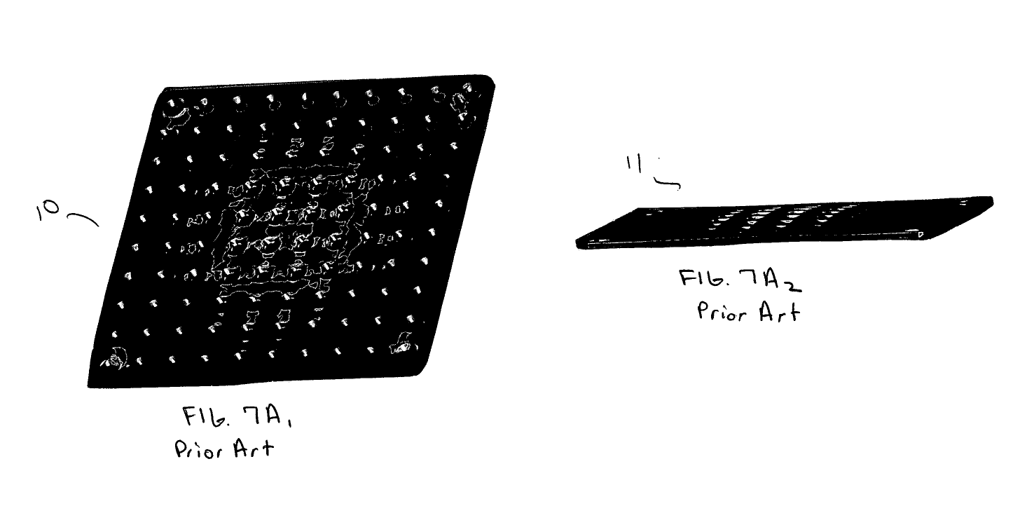

In designing the different connectors 5 it is advantageous to avoid the formation of sharp stress concentrations during the stretching as well as compression of the connectors 5. FIGS. 5A-J show examples of different connectors, which can be used to achieve sufficient deformation behavior of bioabsorbable, polymeric meshes of the present invention. In FIGS. 5A-J, each connector 5 connects openings 6. FIGS. 6A-K show FEM-modeling of different connector geometry's. FIGS. 6A-K each consist of three images in which 7 is the initial state of the connector, 8 is the stretched state of the connector, and 9 is the compressed state of the connector. FIG. 6A illustrates a high stress concentration in the middle of the prior art connector used in metallic meshes, whereas FIGS. 6B-6K show better stress distributions over larger areas for different connector embodiments of the present invention. To FEM model the stresses, two connectors were fastened through the openings in either end of the connector. Both ends were then stretched and compressed with the same force. In FIG. 6A, the stresses are concentrated on a very small area of the prior art connector, specifically in the middle of the prior art connector. If this connector were made of a polymer material, a crack would likely be initiated at the focus of the stress, regardless of whether the material itself would be deformable. In FIGS. 6B-K the stresses are distributed on a much larger area and meshes formed from theses connectors can be stretched or compressed along a larger continuum without the risk of breakage.

FIG. 4 show both an embodiment of the bioabsorbable, polymeric mesh 4A of the present invention and a plate 4B, both deformed to fit a curved bone surface. The mesh and plate are both approximately 51×51×0.8 mm and both are biaxially oriented. Mesh 4A weights approximately 157 g and plate 4B weighs approximately 2.14 g. Mesh 4A has 26.6% less polymer than plate 4B. As shown, even with the same inherent material characteristics as the present invention, 4B cannot be deformed to fit a spherical surface exactly because of the plate geometry. For example, the prior art plate 4B has wavy edges, which would not allow the plate 4B to lie flush with the bone. This inability to deform adequately is caused by the inability to compress portions of the plate 4B.

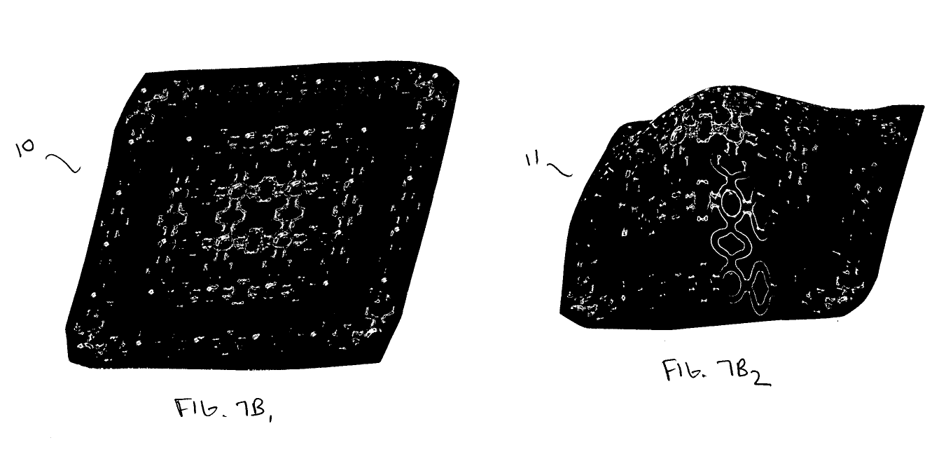

The stress distributions associated with a mesh as compared to a plate are shown in FIGS. 7A-B. FIGS. 7A-B show FEM-modeling of a prior art plate (in FIG. 7A) and an embodiment of the bioabsorbable, polymeric mesh of the present invention (in FIG. 7B). The prior art plate shown in FIG. 7A is biaxially oriented and the bioabsorbable, polymeric mesh has the geometry shown in FIG. 3B. Outer edges of the prior art plate and the bioabsorbable, polymeric mesh were each fastened to a metallic ring with an inner hole diameter of 69 mm. A force of 1-5 N was applied to the center of the prior art plate and the bioabsorbable mesh. FIGS. 7A-7B show two images in which 10 represents the initial state of the plate or mesh and 11 represents the deformed state. The prior art plate was displaced from approximately 1-4 mm and the bioabsorbable, polymeric mesh was displaced approximately 26-39 mm. As illustrated, the deformation that occurs is significantly higher in the case of the bioabsorbable, polymeric mesh according to the present invention, than in the case of the prior art plate.

The open structure of the bioabsorbable, polymeric mesh also significantly reduces the total implanted mass of the bioabsorbable, polymeric mesh implant thereby avoiding foreign body reactions during the degradation of the bioabsorbable, polymeric mesh implant. The influence of implanted mass on foreign body reactions is reviewed by Rozema et al. in Resorbable poly(L-lactide) Bone Plates and Screws: Tests and Applications, Doctoral Thesis, Groningen University, Groningen, Netherlands, 1991, p. 61-78, the disclosure hereby incorporated by reference, in its entirety.

In addition, the open structure of the bioabsorbable, polymeric mesh implant according to the present invention allows for easy fastening of the implant by suturing, which is especially favourable, when performing cranioplasties in the case of growth disturbances in young individuals. Often young bones can be too weak for normal fasteners, like screws, and the implant must be fastened to many locations on a large area to secure the fixation.

The deformation behaviour and open structure of the bioabsorbable, polymeric mesh makes cutting of the bioabsorbable, polymeric mesh very easy. In operating rooms, normal scissors or plungers can be used to cut away areas of the bioabsorbable, polymeric mesh implant, which are not needed for secure fixation of the bones.

In an embodiment of the present invention, growth of soft tissue through any openings of the bioabsorbable, polymeric mesh is not desired (see e.g. H. Peltoniemi “Biocompatibility and Fixation Properties of Absorbable Miniplates and Screws in Growing Calvarium”, Doctoral Thesis, Helsinki University, Helsinki, Finland, 2000, p. 50, the enclosure of which is incorporated by reference, in its entirety). In this embodiment, films, such as a non-woven fabric, a woven fabric or a membrane, made of the same or another bioabsorbable, biocompatible, deformable or rubber-like material, can be attached to one or two surfaces of the bioabsorbable, polymeric mesh implant. The film can be relatively thin and is attached to the bioabsorbable, polymeric mesh by any known means, such as heat, compression molding or by means of a bioabsorbable, biocompatible adhesive. In an embodiment of the mesh including the film, the film is continuous and impermeable, impeding liquids, cells and/or other components from passing through the bioabsorbable, polymeric mesh implant. In another embodiment of the bioabsorbable, polymeric mesh implant, the mesh includes a film, the film can have holes or cavities of a specific diameter and form, to selectively allow some components to pass through the bioabsorbable, polymeric mesh implant.

The bioabsorbable, polymeric mesh implant of the present invention can be used to heal bone injuries, particularly injuries to the skull. In order to use the bioabsorbable, polymeric mesh implant of the present invention, a surgeon first removes loose bone fragments from the injured area. Next, a bioabsorbable, polymeric mesh implant is removed from a sterile package and initially placed near the wound in order to determine if the mesh needs to be made smaller. If the bioabsorbable, polymeric mesh does need to be adjusted, the surgeon cuts the bioabsorbable, polymeric mesh with scissors until it is the correct size. Following cutting the bioabsorbable, polymeric mesh, the surgeon deforms the bioabsorbable, polymeric mesh so that it will fill the injured area exactly. The deformation can include stretching, compressing, and bending the bioabsorbable, polymeric mesh at room temperature so that it will fit the injury exactly. Next, the surgeon can attach the larger bone fragments to the bioabsorbable, polymeric mesh with bioabsorbable fasteners, such as screws or tacks, through the openings in the bioabsorbable, polymeric mesh. If there are smaller or weaker fragments, the surgeon can attach them to the bioabsorbable, polymeric mesh by sutures. After attaching the bone fragments (if there are any) the bioabsorbable, polymeric mesh is placed over the injured area and fastened securely to the bone with bioabsorbable fasteners, such as screws or tacks.

During the healing process, the bone fragments are securely fixed at the location the surgeon placed them. Over time the bioabsorbable, polymeric mesh will gradually lose strength and ultimately degrade within approximately one to three years. The degraded material will be totally absorbed through the normal metabolism of the patient.

### EXAMPLE 1

The bioabsorbable, polymeric mesh and method for using the bioabsorbable, polymeric mesh can be used to treat a patient with a comminuted fracture in the prefrontal area of the skull. In this embodiment, a surgeon needs to fix the bone fragments in their original pre-trauma locations during the healing period. First, the surgeon removes loose bone fragments from the damaged area. Then a bioabsorbable, polymeric mesh implant approximately 51×51×0,6 mm, similar to that shown in FIG. 4 is taken out of a sterile package and handed to the surgeon. The surgeon will first cut out one or two edges of the bioabsorbable, polymeric mesh, depending on whether the trauma area is smaller than the bioabsorbable, polymeric mesh implant. The cutting is performed with normal scissors. After cutting, the surgeon will start to deform the bioabsorbable, polymeric mesh in order to fit it the damaged area by stretching the middle of the bioabsorbable, polymeric mesh and compressing the outer area of the bioabsorbable, polymeric mesh implant to achieve a spherical shape. When the proper shape of the bioabsorbable, polymeric mesh is achieved, the surgeon has completed the deformation phase.

Thereafter the surgeon fastens some of the bigger loose bone fragments to the bioabsorbable, polymeric mesh implant with bioabsorbable screws or tacks by using the openings in the bioabsorbable, polymeric mesh and some smaller and weaker fragments by suturing them to the bioabsorbable, polymeric mesh implant.

Then the surgeon places the deformed bioabsorbable, polymeric mesh implant with the bone fragments fastened to it on the damaged area and fastens it securely to the undamaged cranial bone around the damaged area with bioabsorbable screws or tacks through the openings in the bioabsorbable, polymeric mesh.

During the healing period of approximately 6 weeks, the bone fragments will be securely fastened at the locations the surgeon has placed them during the operation. After approximately 6 weeks, the trauma will be well healed and the bioabsorbable, polymeric mesh implant can start gradually to loose its strength. The bioabsorbable, polymeric mesh implant will be completely degraded in approximately one to three years and the degraded products will be completely absorbed through the normal metabolism of the patient.

1. A bone fixation bioabsorbable polymeric mesh implant for the fixation of bone fragments and bridging of bone defects or gaps comprising:
a bone fixation bioabsorbable polymeric mesh including a plurality of more than twenty openings and connectors, wherein each opening is connected to another opening by a connector;
and wherein the bone fixation bioabsorbable polymeric mesh is deformable at room temperature without breaking and is capable of deforming to fit a curved bone surface and lie flush with the bone surface.

2. (canceled)

3. (canceled)

4. The bioabsorbable polymeric mesh implant of claim 1, wherein the bioabsorbable polymeric mesh is manufactured of a nonoriented polymer.

5. The bioabsorbable polymeric mesh implant of claim 1, wherein the bioabsorbable polymeric mesh is manufactured of a uni-axially oriented polymer.

6. The bioabsorbable polymeric mesh implant of claim 1, wherein the bioabsorbable polymeric mesh is manufactured of a biaxially oriented polymer.

7. The bioabsorbable polymeric mesh implant of claim 1, reinforced with resorbable fibers.4

8. The bioabsorbable polymeric mesh implant of claim 7, wherein the fibers comprise resorbable polymeric fibers, or biodegradable glass fibers.

9. The bioabsorbable polymeric mesh of implant of claim 8, wherein the biodegradable glass fibers comprise b-tricalsiumphosphate fibres, bio-glass fibres or CaM fibres.

10. The bioabsorbable polymeric mesh implant of claim 1, including ceramic powder to promote bone growth.

11. The bioabsorbable polymeric mesh implant of claim 1, including drugs.

12. The bioabsorbable polymeric mesh implant of claim 1, which can be deformed with scissors or plungers at room temperature.

13. The bioabsorbable polymeric mesh implant of claim 1, wherein the bioabsorbable polymeric mesh is attachable to bone by bioabsorbable fasteners.

14. The bioabsorbable polymeric mesh implant of claim 13, wherein the bioabsorbable fasteners include screws, tacks, or sutures.

15. The bioabsorbable polymeric mesh implant of claim 1, wherein the connectors can be used as fastener openings.

16. A bone fixation bioabsorbable polymeric mesh implant for the fixation of bone fragments and bridging of bone defects or gaps comprising:
a bone fixation bioabsorbable polymeric mesh including a plurality of more than twenty openings and connectors, wherein each opening is connected to another opening by a connector and wherein the mesh has a first and second surface; and
a bioabsorbable film attached along either the first or second surface of the mesh;
wherein the bone fixation bioabsorbable polymeric mesh with the bioabsorbable film is deformable at room temperature without breaking and is capable of deforming to fit a curved bone surface and lie flush with the bone surface.

17. The bioabsorbable polymeric mesh implant of claim 16, wherein the bioabsorbable film comprises a woven fabric, a non-woven fabric, or rubber-like material.

18. The bioabsorbable polymeric mesh implant of claim 16, wherein the bioabsorbable film includes bioactive components or drugs.

19. The bioabsorbable polymeric mesh implant of claim 16, wherein the bioabsorbable film is porous and the pores allow selective transport of components through the bioabsorbable film.

20. A method of treating a bone injury comprising the steps of:
applying the bioabsorbable polymeric mesh implant of claim 1 to a damaged bone area, the damaged bone area being curved, concave, convex, angular, spherical, or any combination thereof.

21. A method of treating a bone injury comprising the steps of:
applying the bioabsorbable polymeric mesh implant of claim 14 to a damaged bone area, the damaged bone area being curved, concave, convex, angular, spherical, or any combination thereof.

22. A method of treating a bone injury comprising the steps of:
deforming the bioabsorbable polymeric mesh of claim 1 at room temperature;
applying the deformed bioabsorbable polymeric mesh to the bone injury.

23. The method of claim 22, where during the deformation step the openings of the bioabsorbable polymeric mesh are not deformed.

24. The method of claim 22, where the deforming step includes stretching, compressing, contouring, or any combination thereof.

25. A method of treating a bone injury comprising the steps of:
deforming the bioabsorbable polymeric mesh of claim 14 at room temperature;
applying the deformed bioabsorbable polymeric mesh to the bone injury.

26. The method of claim 25, where during the deformation step the openings of the bioabsorbable polymeric mesh are not deformed.

27. The method of claim 25, where the deforming step includes stretching, compressing, contouring, or any combination thereof.

28. A system for fastening bone fragments comprising:
a bioabsorbable, polymeric mesh implant, according to claim 1; and
bioabsorbable fasteners.

29. The system of claim 28, wherein the bioabsorbable fasteners comprise screws, tacks, sutures, or combinations thereof.

30. The bioabsorbable polymeric mesh implant of claim 1, wherein the bioabsorbable polymeric mesh comprises a polymer, copolymer, polymer alloy, composite, or combination thereof.

31. The bioabsorbable polymeric mesh implant of claim 1, wherein the bioabsorbable polymeric mesh comprises poly-α-hydroxy acids and other aliphatic bioabsorbable polyesters, polyanhydrides, polyorthoesters, polyorganophosphatzenes, tyrosine polycarbonates, or other bioabsorbable polymers.
